(19) United States
(12) Patent Application Publication
(10) Pub. No.: US 2006/0228804 A1
(43) Pub. Date: Oct. 12, 2006

(54) Modified ruthenium complex luminescence dye for oxygen sensing

(75) Inventors: Joshua Xu, Irvine, CA (US); Chang-Dong Feng, Long Beach, CA (US)

Correspondence Address: WESTMAN CHAMPLIN & KELLY, P.A., SUITE 1400, 900 SECOND AVENUE SOUTH, MINNEAPOLIS, MN, 55402-3319, US

(73) Assignee: Rosemount Analytical Inc., Irvine, CA (US)

(21) Appl. No.: 11/399,745

(22) Filed: Apr. 7, 2006

Related U.S. Application Data
(60) Provisional application No. 60/669,574, filed on Apr. 8, 2005.

(51) Int. Cl. G01N 33/00

(52) U.S. Cl. 436/84

(57) ABSTRACT

Modification of ruthenium complex luminescence dye for oxygen sensing is provided. Generally, modification includes bonding long chain hydrophobic organic groups to the ligands of the ruthenium complex in order to increase solubility of the ruthenium complex in non-polar organic solvents. A sensor manufacture using the modified ruthenium complex luminescence dye is also provided.

### CROSS-REFERENCE TO RELATED APPLICATION

The present application is based on and claims the benefit of U.S. provisional patent application Ser. No. 60/669,574, filed Apr. 8, 2005, the content of which is hereby incorporated by reference in its entirety.

### BACKGROUND OF THE INVENTION

Oxygen is a gas of significant interest, simply because of its role in the cycle of all living organisms. Measurement of oxygen concentration or partial pressure is important in a wide variety of applications. In some applications, gaseous oxygen concentrations are measured directly. In other applications, the concentration of oxygen dissolved in a liquid is measured. It is important to realize that the term “dissolved oxygen” refers to gaseous oxygen dissolved in water, and it should not be confused with combined oxygen as found in the water molecule, H₂O.

Dissolved oxygen measurement is very important in the treatment of domestic wastewater, as well as industrial waste from such sources as food, pulp and paper, chemical, and metal industries. Most water pollutants from these sources fall into one of two categories: (1) those that cannot be further broken down but persist in or out of solution; and (2) those that are biologically degradable. Biologically degradable pollutants are both organic and inorganic degradable substances, of which the organic type tends to represent a large majority.

The primary function of dissolved oxygen in a waste stream is to enhance the oxidation process by providing oxygen to aerobic bacteria so that they will be able to successfully perform their function of turning organic wastes into their inorganic byproducts, specifically, carbon dioxide, water, and sludge. This oxidation process, known as the activated sludge process, is probably the most popular and widely used method of secondary waste treatment today and is employed downstream of a primary settling tank. The process takes place in an aeration basin and is accomplished by aeration (the bubbling of air or pure oxygen through the waste water at this point in the treatment process). In this manner, the oxygen, which is depleted by the bacteria, is replenished to allow the process to continue.

In order to keep the waste treatment process functioning properly, a certain amount of care must be taken to hold the dissolved oxygen level within an acceptable range and to avoid conditions detrimental to the process. It is also important to make the measurement at a representative location on a continuous basis to have a truly instantaneous measurement of the biological activity taking place in the aeration basin.

Yet another promising application for the measurement of dissolved oxygen is in biological specimens. These biological specimens may be in vitro specimens in a laboratory, or in vivo specimens within a patient. The measurement of dissolved oxygen in biological specimens provides important diagnostic information for care providers, and/or information about the efficacy of a particular treatment.

Traditionally, dissolved oxygen can be measured in a variety of ways. For example, various laboratory methods exist, such as the Winkler Method; electrochemical analysis, such as conductimetric, voltimetric, and galvanic; and membrane electrode methods (galvanic membrane electrodes and ampierometric membrane electrodes). Yet another way in which oxygen, whether dissolved in a liquid, or gaseous, can be measured is by employing optical techniques. For example, known oxygen sensors employ a ruthenium complex luminescence dye which luminescences in the presence of oxygen. Measurement of the luminescence provides an indication of oxygen concentration.

Generally, sensors that measure dissolved oxygen in liquid and sensors that measure gaseous oxygen are of significantly different designs. However, since embodiments of the present invention are applicable to both the measurement of gaseous oxygen and dissolved oxygen in liquid, both types of situations are presented here to provide a better understanding of the vast array of potential applications for various embodiments.

Commercially available ruthenium complex luminescence dyes used for oxygen sensing are generally hydrophilic and have low solubility in non-polar polymers. The degree to which commercially available ruthenium complex luminescence dye is hydrophilic limits the type of media which can be used to immobilize the dye. For example, in most applications that use ruthenium complex dye for oxygen sensing, the dye is dissolved in silica-based sol solution, and then a thin film is cast through a known sol-gel process from the sol solution. However the coating process of sol-gel silica involves many chemical changes and is sensitive to variations in temperature and humidity. Moreover, the quality control of the sol-gel coating is difficult. Providing a ruthenium complex luminescence dye that was not limited to sol-based media for immobilization would facilitate simpler manufacture. Moreover, removing the stringent quality control requirements of sol-gel coating would further facilitate yields and potentially reduce costs.

### SUMMARY

Modification of ruthenium complex luminescence dye for oxygen sensing is provided. Generally, modification includes bonding long chain hydrophobic organic groups to the ligands of the ruthenium complex in order to increase solubility of the ruthenium complex in non-polar organic solvents. A sensor manufacture using the modified ruthenium complex luminescence dye is also provided.

### BRIEF DESCRIPTION OF THE DRAWINGS

FIG. 1 is a diagrammatic view of a ruthenium complex luminescent dye molecule in accordance with the prior art.

FIG. 2 is a diagrammatic view of a ruthenium complex luminescence dye molecule in accordance with an embodiment of the present invention.

FIG. 3 is a diagrammatic view of an oxygen sensor employing a modified ruthenium complex luminescence dye in accordance with an embodiment of the present invention.

### DETAILED DESCRIPTION

Embodiments of the present invention generally provide modification of a ruthenium complex luminescence dye for improved solubility in non-polar polymers. The modification is generally done by covalently attaching hydrophobic organic groups to the ligands in the ruthenium complex. The chemically modified ruthenium complex has higher solubility in hydrophobic polymeric media. The resulting polymeric coating has a more uniform distribution of the luminescence dye. The increased solubility of the ruthenium complex in the hydrophobic polymer medium allows the ruthenium complex to be more soluble in non-polar organic solvents such as toluene. Further, the increased solubility of the ruthenium complex luminescence dye facilitates the use of polymers as the immobilization media for the modified ruthenium complex luminescence dye. There are many advantages to using a polymer as the immobilization media instead of sol-gel derived silica. Polymer coatings will not experience: the shrinkage and pore collapse which are usually observed in sol-gel derived silica. Moreover, the processing of polymer coatings, on the other hand, does not involve the many chemical changes, in comparison to sol-gel silica processing, and it is easy to control.

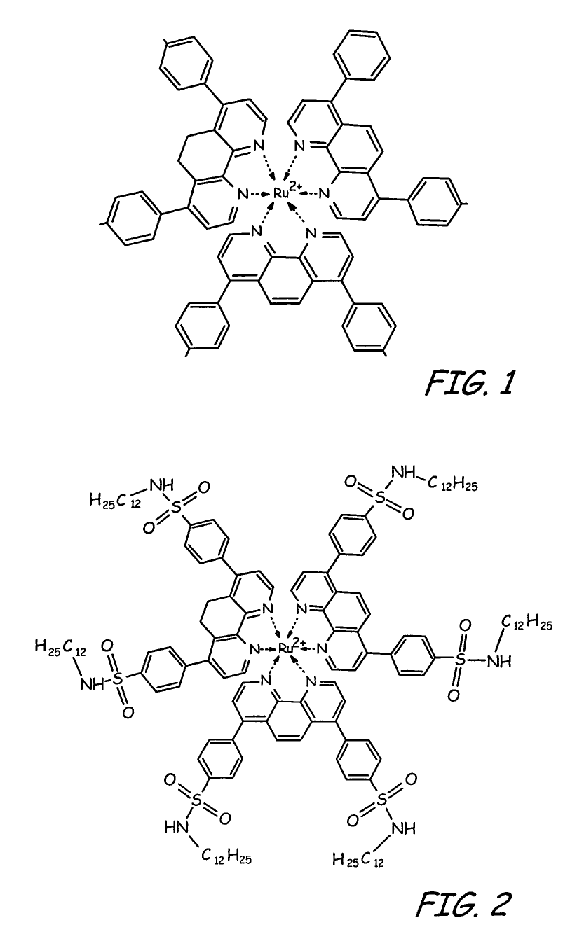

FIG. 1 is a diagrammatic view of a typical ruthenium complex Ru(II)-tris(4,7-diphenyl-1,10-phenanthroline). The complex itself is a relatively small cation and has relative low solubility in non-polar organic solvents, such as toluene or acetone.

FIG. 2 is a diagrammatic view of a chemically modified ruthenium complex dye in accordance an embodiment of the present invention. FIG. 2 shows modified Ru(II)-tris(4,7-diphenyl-1,10-phenanthroline). An important feature illustrated in FIG. 2 of the modification is the addition of six C₁₂H₂₅ hydrocarbon chains. In FIG. 2, the long chain hydrophobic organic groups are illustrated as C₁₂H₂₅ but in reality can be any long chain hydrophobic organic group, such as a hydrocarbon chain of suitable length, to provide the requisite degree of solubility in non-polar organic solvents. For the purposes of this patent document, “long chain hydrophobic organic group” is intended to mean any chain of six or more hydrophobic organic groups. Preferably, the long chain hydrophobic organic groups are long hydrocarbon chains that are bonded covalently to the ligands in the ruthenium complex.

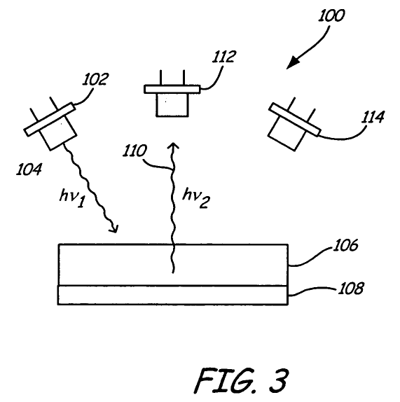

FIG. 3 is a diagrammatic view of an optical oxygen sensor 100 in accordance with an embodiment of the present invention. Sensor 100 includes excitation source 102 that is illustrated as a light emitting diode, but may be any suitable excitation source. Source 102 generates excitation illumination 104 that passes through transparent substrate 106 and interacts with sensing layer 108. In accordance with an embodiment of the present invention, sensing layer 108 is comprised of a modified ruthenium complex dye having long chain hydrophobic organic groups covalently bonded to the ligands of the ruthenium complex. Additionally, sensing layer 108 provides the ruthenium dye immobilized on a polymer, such as polystyrene. In accordance with known techniques, the excitation illumination interacts with the sensing layer and luminescences as indicated at reference numeral 110. The luminescence illumination is sensed by luminescent light detector 112 which is used to measure the luminescence and ultimately provide an indication of oxygen concentration or partial pressure. Excitation light source 102 emits light h₁ which excites the modified ruthenium complex luminescence dye molecules in sensing layer 108. The excited dye molecules then emit luminescent light h₂, which is then measured by luminescent light detector 112. Sensor 100 also generally includes excitation light detector 114 which is used to measure characteristics of the excitation illumination in order to compensate for changes therein.

Although the present invention has been described with reference to preferred embodiments, workers skilled in the art will recognize that changes may be made in form and detail without departing from the spirit and scope of the invention.

What is claimed is:

1. A ruthenium complex luminescent dye for sensing oxygen, the dye being comprised of molecules of Ru(II)-tris(4,7-diphenyl-1,10phenanthroline), with a long chain hydrophobic group attached to each ligand thereof.

2. The ruthenium complex luminescent dye of claim 1, wherein each long chain hydrophobic organic group is attached to each ligand by covalent bonds.

3. The ruthenium complex luminescent dye of claim 1, wherein each long chain hydrophobic organic group includes a long hydrocarbon chain.

4. The ruthenium complex luminescent dye of claim 3, wherein each long hydrocarbon chain includes C₁₂H₂₅.

5. The ruthenium complex luminescent dye of claim 1, wherein the dye is dissolved in a non-polar organic solvent.

6. The ruthenium complex luminescent dye of claim 5, wherein the solvent is toluene.

7. An optical oxygen sensor comprising:
a source of excitation illumination;
a sensing layer disposed to receive the excitation illumination and generate luminescence illumination based on a concentration of oxygen proximate the sensing layer;
a luminescence sensor disposed to measure the luminescence illumination to provide an indication of the oxygen concentration; and
wherein the sensing layer includes a modified ruthenium complex dye having long chain hydrophobic organic groups.

8. The optical oxygen sensor of claim 7, wherein the ruthenium complex luminescence dye is immobilized on a polymer.

9. The optical oxygen sensor of claim 7, wherein the ruthenium complex dye is comprised of molecules of Ru(II)-tris(4,7-diphenyl-1,10phenanthroline), with a long chain hydrophobic organic group attached to each ligand thereof.

10. The optical oxygen sensor of claim 9, wherein each long chain is covalently bonded to a respective ligand.

11. The optical oxygen sensor of claim 9, wherein each long chain hydrophobic organic group includes at least six carbon atoms.

12. The optical oxygen sensor of claim 11, wherein each long chain hydrophobic organic group includes C₁₂H₂₅.
